(19) United States
(12) Patent Application Publication
(10) Pub. No.: US 2012/0020988 A1
(43) Pub. Date: Jan. 26, 2012

(54) Antibodies specifically binding to human TSLPR and methods of use

(75) Inventors: JOHANNES AUER, SCHWAIGEN (DE); MARIA FUENTES, WOODCLIFF LAKE, NJ (US); GUY GEORGES, HABACH (DE); HUBERT KETTENBERGER, MUENCHEN (DE); HANS-WILLI KRELL, PENZBERG (DE); JENS NIEWOEHNER, MUENCHEN (DE); GEORG TIEFENTHALER, SINDELSDORF (DE)

(73) Assignee: HOFFMANN-LA ROCHE INC., NUTLEY, NJ (US)

(21) Appl. No.: 13/184,046

(22) Filed: Jul. 15, 2011

(30) Foreign Application Priority Data
Jul. 15, 2010 (EP) 10169728.2

(51) Int. Cl. A61K 39/395; C12N 15/63; C12N 1/21; C12P 21/06; C12N 5/10; C12N 1/19; C07K 16/28; A61P 37/00

(52) U.S. Cl. 424/172.1; 530/389.1; 530/387.3; 536/23.53; 435/320.1; 435/69.6; 435/325; 435/358; 435/352; 435/365; 435/254.2; 435/252.33

(57) ABSTRACT

An antibody specifically binding to human thymic stromal lymphopoietin receptor (TSLPR), characterized in comprising as heavy chain variable domain CDR regions a CDR1 region of SEQ ID NO:2 or 17, a CDR2 region of SEQ ID NO:3 or 10, and CDR3 region of SEQ ID NO:4, and as light chain variable domain CDR regions a CDR1 region of SEQ ID NO:6 or 12, a CDR2 region of SEQ ID NO:7, 13 or 15, and a CDR3 region of SEQ ID NO:8. is useful for the treatment of immunological diseases.

### RELATED APPLICATIONS

This application claims of priority under 35 USC §119(a) to European patent application number 10169728.2, filed Jul. 15, 2010, the contents of which are incorporated herein by reference.

### FIELD OF THE INVENTION

The present invention relates to antibodies specifically binding to human TSLPR (TSLPR antibodies), methods for their production, pharmaceutical compositions containing said antibodies, and uses thereof.

### BACKGROUND OF THE INVENTION

Thymic stromal lymphopoietin receptor (TSLPR, TSLP receptor, CLRF2, SwissProt Q9HC73) is a Type I cytokine receptor subunit which is closely related to the common cytokine γ chain (γc). TSLP is an interleukin (IL)-7-like cytokine that initiates and propagates allergic immune responses. TSLP is produced predominantly by epithelial cells and activated mast cells, and stimulates myeloid dendritic cells (mDC), which express a heterodimer consisting of the interleukin 7 (IL-7) alpha chain and TSLP receptor. TSLP-activated mDC can promote naive CD4+ T cells to differentiate into a Th2 phenotype and can promote the expansion of CD4+ Th2 memory cells.

Human TSLP receptor is mentioned as “Cytokine Receptor Common Gamma Chain Like” in WO 99/47538 (U.S. Pat. No. 6,844,170, U.S. Pat. No. 6,861,227, U.S. Pat. No. 6,982,320) and WO 2001/12672. Human TSLP receptor is also mentioned in Pandey, A., et al., Nature Immunol. 1 (2000) 59-64, Soumelis, V., et al., Nature Immunol. 3 (2002) 673-680, Soumelis, V., et al., Springer Sem. Immunopath. 25 (2004) 325-333, WO 2002/068646 (U.S. Pat. No. 6,890,734, U.S. Pat. No. 7,071,308), WO 2003/065985, WO 2002/00723, (U.S. Pat. No. 6,955,895), WO 2002/00724, WO 99/47538, (U.S. Pat. No. 6,844,170, U.S. Pat. No. 6,861,227, U.S. Pat. No. 6,982,320), US 2005/249712 and WO 2006/023791. Antibodies against TSLP receptor are also mentioned in WO 2007/112146 and WO 2009/100324.

### SUMMARY OF THE INVENTION

The invention comprises an antibody that specifically binds to TSLPR. In one aspect, the antibody comprises a heavy chain variable domain comprising a CDR1 region of SEQ ID NO: 2, a CDR2 region of SEQ ID NO:3, and CDR3 region of SEQ ID NO:4, and a light chain variable domain comprising a CDR1 region of SEQ ID NO: 6, a CDR2 region of SEQ ID NO:7, and a CDR3 region of SEQ ID NO:8, or a humanized variant thereof. In an embodiment, the antibody is a humanized variant.

In one aspect, the invention comprises an antibody specifically binding to TSLPR, characterized in that the heavy chain variable domain comprises SEQ ID NO:1 and the light chain variable domain comprises SEQ ID NO:5. In a further embodiment, the antibody is a humanized variant.

In one aspect, the invention comprises an antibody specifically binding to TSLPR, characterized in being a chimeric or humanized variant thereof. In one embodiment, the variant is characterized in comprising as heavy chain variable domain CDR regions a CDR1 region of SEQ ID NO: 2 or 17, a CDR2 region of SEQ ID NO:3 or 10, and CDR3 region of SEQ ID NO:4, and as light chain variable domain CDR regions a CDR1 region of SEQ ID NO: 6 or 12, a CDR2 region of SEQ ID NO:7, 13 or 15, and a CDR3 region of SEQ ID NO:8. In a further embodiment, the antibody is a humanized variant.

In one aspect, the humanized antibody comprises
a) as heavy chain variable domain CDR regions a CDR1 region of SEQ ID NO:2, a CDR2 region of SEQ ID NO:10, and CDR3 region of SEQ ID NO:4, and as light chain variable domain CDR regions a CDR1 region of SEQ ID NO:12, a CDR2 region of SEQ ID NO:13 and a CDR3 region of SEQ ID NO:8, or
b) as heavy chain variable domain CDR regions a CDR1 region of SEQ ID NO:2, a CDR2 region of SEQ ID NO:10, and CDR3 region of SEQ ID NO:4, and as light chain variable domain CDR regions a CDR1 region of SEQ ID NO:12, a CDR2 region of SEQ ID NO:15 and a CDR3 region of SEQ ID NO:8, or
c) as heavy chain variable domain CDR regions a CDR1 region of SEQ ID NO:17, a CDR2 region of SEQ ID NO:10, and CDR3 region of SEQ ID NO:4, and as light chain variable domain CDR regions a CDR1 region of SEQ ID NO:12, a CDR2 region of SEQ ID NO:15 and a CDR3 region of SEQ ID NO:8, or
d) as heavy chain variable domain CDR regions a CDR1 region of SEQ ID NO:2, a CDR2 region of SEQ ID NO:10, and CDR3 region of SEQ ID NO:4, and as light chain variable domain CDR regions a CDR1 region of SEQ ID NO:12, a CDR2 region of SEQ ID NO:7 and a CDR3 region of SEQ ID NO:8.

In one embodiment, the humanized antibody comprises
a) the heavy chain variable domain comprises SEQ ID NO:9 and the light chain variable domain comprises SEQ ID NO:11, or
b) the heavy chain variable domain comprises SEQ ID NO:9 and the light chain variable domain comprises SEQ ID NO:14, or
c) the heavy chain variable domain comprises SEQ ID NO:16 and the light chain variable domain comprises SEQ ID NO:18, or
d) the heavy chain variable domain comprises SEQ ID NO:9 and the light chain variable domain comprises SEQ ID NO:19.

In an aspect, the antibody is humanized or is a human antibody. In an embodiment, the antibody is of human kappa isotype. In one embodiment, the antibody preferably comprises a Fc part derived from human origin. In an embodiment, the antibody is of human IgG1 or IgG4 isotype. In an embodiment, the antibody is of human IgG4(S228P,L235E) class. In an embodiment, the antibody is characterized in that the heavy chain variable domain is a CDR grafted IgG4(S228P,L235E) subtype form of heavy chain variable domain and the light chain variable domain is a CDR grafted kappa isotype form of light chain variable domain. An example of IgG4(S228P,L235E) constant chain is provided in the sequence listing below (SEQ ID NO:22). IgG4(S228P,L235E) means IgG4 with a substitution of serine 228 to proline and lysine 235 to glutamic acid. IgG1(L234A,L235A), which is the same as IgG1 (LALA), has to be understood accordingly.

In an embodiment, the antibody is preferably characterized in that non-binding of the antibody to complement factor C1q refers to an ELISA assay measurement wherein the maximal binding (Bmax) of the antibody at a concentration of 10 μg/ml and a molecular weight of 150.000 to C1q is 30% or lower, preferably 20% or lower compared to Bmax of a chimeric antibody consisting of the variable regions of TSLP-012 and human light chain kappa region SEQ ID NO:23 and human heavy chain IgG1 region SEQ ID NO:20 (Chimeric Mab TSLPR-012).

In an embodiment, the antibody is preferably characterized in that non-binding of the antibody to Fcγ receptor on NK cells refers to assay wherein the maximal binding (Bmax) of the antibody at a concentration of 20 μg/ml to NK cells is 20% or lower, preferably 10% or lower compared to Bmax of antibody Chimeric Mab TSLPR-012.

In an embodiment, the antibody is preferably characterized in that it does not bind to FcγRI. This means that the antibody is characterized by an EC50 value which is five fold or more, preferably seven fold or more, such as eight fold or more compared to the EC50 value of Chimeric Mab TSLPR-012, when measured in an assay testing binding of the antibody in a concentration ranging from 0.078-10 μg/ml to a B-cell lymphoma cell lacking FcγRIIA and FcγIIB, but expressing recombinant FcγRI.

In an embodiment, the antibody is preferably characterized as being an IgG4 antibody or an IgG1 antibody comprising at least one amino acid mutation, preferably in the human Fc part, causing non-binding to complement factor C1q and/or non-binding to human Fcγ receptor on NK cells.

In an embodiment, the antibody is preferably characterized by being of human subclass IgG4. In a further embodiment of the invention, the antibody is characterized by being of any IgG class, preferably being IgG1 or IgG4, containing at least one mutation in E233, L234, L235, G236, D270, N297, E318, K320, K322, A327, A330, P331 and/or P329 (numbering according to EU index). In yet further embodiment, the IgG1 mutations PVA236 (PVA236 means that the amino acid sequence ELLG (given in one letter amino acid code) from amino acid position 233 to 236 of IgG1 or EFLG of IgG4 is replaced by PVA), L234A,L235A and/or GLPSS331 (GLPSS331 means that in the 331 region ALPAP of IgG1 or GLPAP of IgG2 is changed to GLPSS) as well as the IgG4 mutation L235E. In an embodiment, the antibody of IgG4 subclass contains the mutation S228P or the mutation S228P and L235E (Angal, S., et al., Mol. Immunol. 30 (1993) 105-108).

In an embodiment, the antibody is of human subclass IgG4, containing one or more mutation(s) from PVA236, GLPSS331 and/or of human IgG1 subclass with mutations L234A,L235A (LALA mutation), numbering according to EU index).

In an embodiment, the antibody specifically binds to TSLPR, is an IgG1 class, preferably comprising as γ heavy chain SEQ ID NO:20, optionally comprising mutation L234A,L235A.

In an aspect, the antibody is an IgG4. In an embodiment, the antibody comprises the S228P and/or L235E mutation(s). In an embodiment, the antibody comprises a heavy chain constant region of SEQ ID NO:21, optionally comprising mutations S228P and/or L235E, and comprises further preferably a light chain constant region of SEQ ID NO:23. In an embodiment, the constant regions of the antibody according the inventions are of SEQ ID NO:21 and 22.

In one aspect, the antibody is preferably characterized in that it does not elicit complement-dependent cytotoxicity (CDC).

In one aspect, the antibody is preferably characterized in that it does not elicit antibody-dependent cellular cytotoxicity (ADCC).

In an embodiment, the anti-TSLPR antibodies or single heavy or light chains characterized by their CDRs, variable regions, complete amino acid sequences or hybridomas and which comprises no Fc part or any type of Fc part.

In an embodiment, the antibodies, are monoclonal antibodies, characterized in that said antibodies specifically bind TSLPR, contains a Fc of human origin and do not bind human complement factor C1q and/or human Fcγ receptor on NK cells, by being of human IgG4 type or of human IgG1 or human IgG4 both modified by the above mentioned mutations.

In an aspect, the antibody specifically binds to human TSLPR on TSLPR-transfected BA/F3 cells (ACC 300, DSMZ, http://www.dsmz.de) with an EC50 value of at least 10⁻⁹ M⁻¹, preferably 10⁻⁹ M⁻¹ to 10⁻¹² M⁻¹ (FACS assay). In an embodiment, the antibody inhibits the BA/F3-TSLPR/IL7Rα proliferation with an IC₅₀ value of 40 ng/ml or lower (based on MW 150.000).

In one embodiment the antibody inhibits dendritic cell (DC) activation and cytokine/chemokine secretion with an IC₅₀ value of 6.0 nM or lower. In one embodiment the antibody inhibits TARC dendritic cell (DC) activation and cytokine/chemokine secretion with an IC₅₀ value of 3.0 nM or lower.

In one embodiment the antibody inhibits Th2 polarization measured as IL-13 cytokine production with an IC₅₀ value of 3.0 nM or lower. In one embodiment the antibody inhibits Th2 polarization measured as IL-5 cytokine production with an IC₅₀ value of 2.0 nM or lower.

In one embodiment the antibody blocks cytokine production in mast cells when stimulated with TSLP and IL-33 or TSLP and TNFα/IL-1β. In one embodiment the antibody blocks IL-13 cytokine production in mast cells when stimulated with TSLP and IL-33 an IC₅₀ value of 0.09 nM, or lower. In one embodiment the antibody blocks IL-5 cytokine production in mast cells when stimulated with TSLP and IL-33 an IC₅₀ value of 0.04 nM, or lower. In one embodiment the antibody blocks GMCSF cytokine production in mast cells when stimulated with TSLP and IL-33 an IC₅₀ value of 0.08 nM, or lower. In one embodiment the antibody blocks IL-13 cytokine production in mast cells when stimulated with TSLP and IL-1β/TNFα an IC₅₀ value of 0.16 nM, or lower. In one embodiment the antibody blocks IL-5 cytokine production in mast cells when stimulated with TSLP and IL-1β/TNFα an IC₅₀ value of 0.13 nM, or lower. In one embodiment the antibody blocks GMCSF cytokine production in mast cells when stimulated with TSLP and IL-1β/TNFα an IC₅₀ value of 0.26 nM, or lower.

In one aspect the antibody is characterized by the above mentioned amino acid sequences or amino acid sequence fragments and properties.

In an aspect, there is provided a nucleic acid encoding an antibody as provided herein. A further embodiment the nucleic acid encodes a heavy and a light chain of an antibody as described herein. In an embodiment, an expression vector comprising a nucleic acid for the expression in a prokaryotic or eukaryotic host cell. of an antibody that specifically binds to TSLPR In an embodiment, there is provided a prokaryotic or eukaryotic host cell comprising a vector. In an aspect, there is provided a method for the production of a recombinant antibody, the method comprises expressing a nucleic acid in a prokaryotic or eukaryotic host cell and recovering said antibody from said cell or the cell culture supernatant.

In one aspect, the antibody is characterized in that the constant chains are of human origin. Such constant chains are well known in the state of the art and described, e.g., by Kabat et al., Sequences of Proteins of Immunological Interest, 5th ed., Public Health Service, National Institutes of Health, Bethesda, Md. (1991). As already mentioned, a useful human light chain constant region comprises an amino acid sequence of a kappa-light chain constant region of SEQ ID NO:23 and a useful human heavy chain constant region comprises SEQ ID NO:20, 21, or 22.

In one aspect, there is provided a pharmaceutical composition. In one embodiment, the pharmaceutical composition comprises an antibody as described herein.

In an aspect, there is provided a method for the manufacture of a pharmaceutical composition comprising an antibody as described herein.

In an aspect, there is provided a method for the manufacture of a medicament for the treatment of a disease, said composition comprising an antibody that specifically binds to human TSLPR as described herein.

In an aspect, there is provided the use of an antibody as described herein for the manufacture of a pharmaceutical composition. The invention further comprises the use of an antibody as described herein for the manufacture of a medicament.

In an aspect, there is provided the use of an antibody as described herein for the treatment of a disease. The invention further comprises the use of an antibody as described herein, wherein the disease is an immunological disease. The invention further comprises an antibody as described herein for use in the treatment of a disease. The invention further comprises an antibody as described herein, wherein the disease is an immunological disease.

In an aspect, there is provided a method for the treatment of a subject suffering from a disease, wherein the treatment comprises administering to the subject an antibody as described herein. The invention further comprises a method for the treatment, characterized in that the disease is an immunological disease. The invention comprises a method for the treatment of a subject in need of therapy, characterized by administering to the subject a therapeutically effective amount of an antibody as described herein.

In an embodiment, the disease to be treated is especially asthma, atopic dermatitis or rheumatoid arthritis. In an embodiment, the disease is mediated by TSLPR activation.

### DESCRIPTION OF THE FIGURES

FIG. 1 Inhibition of Th2 cytokine production in polarization assay with anti-TSLPR antibody TSLPR-012_{—}141. Reference is made to Example 9.

### DETAILED DESCRIPTION OF THE INVENTION

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence. In some embodiments, the VH acceptor human framework is identical in sequence to the VH human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

The term “antibody” encompasses the various forms of antibody structures including, but not being limited to, whole antibodies and antibody fragments. The antibody as described herein is preferably a human antibody, humanized antibody, chimeric antibody, or further genetically engineered antibody as long as the characteristic properties as described herein are retained. “Antibody fragments” comprise a portion of a full length antibody, preferably the variable domain thereof, or at least the antigen binding site thereof. Examples of antibody fragments include diabodies, single-chain antibody molecules, and multispecific antibodies formed from antibody fragments. scFv antibodies are, e.g., described in Huston, J. S., Methods in Enzymol. 203 (1991) 46-88. In addition, antibody fragments comprise single chain polypeptides having the characteristics of a V_{H} domain, namely being able to assemble together with a V_{L} domain, or of a V_{L} domain binding to TSLPR, namely being able to assemble together with a V_{H} domain to a functional antigen binding site and thereby providing the properties of an antibody as described herein. The terms “monoclonal antibody” or “monoclonal antibody composition” as used herein refer to a preparation of antibody molecules of a single amino acid composition.

The term “antigen-binding portion of an antibody” when used herein refers to the amino acid residues of an antibody which are responsible for antigen-binding. The antigen-binding portion of an antibody comprises amino acid residues from the “complementary determining regions” or “CDRs”. “Framework” or “FR” regions are those variable domain regions other than the hypervariable region residues as herein defined. Therefore, the light and heavy chain variable domains of an antibody comprise from N- to C-terminus the domains FR1, CDR1, FR2, CDR2, FR3, CDR3, and FR4. Especially, CDR3 of the heavy chain is the region which contributes most to antigen binding and defines the antibody's properties. CDR and FR regions are determined according to the standard definition of Kabat et al., Sequences of Proteins of Immunological Interest, 5th ed., Public Health Service, National Institutes of Health, Bethesda, Md. (1991).

The term “amino acid” as used within this application denotes the group of naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

The antibodies described herein include, in addition, such antibodies having “conservative sequence modifications” (variant antibodies), nucleotide and amino acid sequence modifications which do not affect or alter the above-mentioned characteristics of the antibody described herein. Modifications can be introduced by standard techniques known in the art, such as site-directed mutagenesis and PCR-mediated mutagenesis. Conservative amino acid substitutions include ones in which the amino acid residue is replaced with an amino acid residue having a similar side chain. Families of amino acid residues having similar side chains have been defined in the art. These families include amino acids with basic side chains (e.g. lysine, arginine, histidine), acidic side chains (e.g. aspartic acid, glutamic acid), uncharged polar side chains (e.g. glycine, asparagine, glutamine, serine, threonine, tyrosine, cysteine, tryptophan), nonpolar side chains (e.g. alanine, valine, leucine, isoleucine, proline, phenylalanine, methionine), beta-branched side chains (e.g. threonine, valine, isoleucine) and aromatic side chains (e.g. tyrosine, phenylalanine, tryptophan, histidine). Thus, a predicted nonessential amino acid residue in a human anti-TSLPR antibody can be preferably replaced with another amino acid residue from the same side chain family. A “variant” anti-TSLPR antibody, refers herein to a molecule which differs in amino acid sequence from a “parent” anti-TSLPR antibody amino acid sequence by up to ten, preferably from about two to about five, additions, deletions and/or substitutions in one or more variable region of the parent antibody. The parent anti-TSLPR antibody is the hamster antibody or chimeric antibody both comprising the VH and VL of the hamster antibody TSLPR-012. Amino acid substitutions can be performed by mutagenesis based upon molecular modeling as described by Riechmann, L., et al., Nature 332 (1988) 323-327 and Queen, C., et al., Proc. Natl. Acad. Sci. USA 86 (1989) 10029-10033.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th ed. Public Health Service, National Institutes of Health, Bethesda, Md., (1991). Thus the expression “amino acid residue positions are numbered according to Kabat” refers to the EU numbering system, also called the EU index, as described in Kabat et al., Sequences of Proteins of Immunological Interest, 5th ed. Public Health Service, National Institutes of Health, Bethesda, Md., (1991).

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, 5th ed., Public Health Service, National Institutes of Health, Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human CDRs and amino acid residues from human FRs. In certain embodiments, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” or “humanized variant” or “humanized antibody” of an antibody, e.g., a non-human antibody, refers to an antibody that has undergone humanization. In one embodiment, one to all six CDRs of an antibody derived from non-human species (e.g. hamster) is (are) grafted into the framework region of a human antibody to prepare the “humanized antibody”. See, e.g., Riechmann, L., et al., Nature 332 (1988) 323-327; and Neuberger, M. S., et al., Nature 314 (1985) 268-270. In one embodiment a humanized variant of an antibody as described herein (which is of non-human origin (e.g. hamster)) refers to an antibody, which is based on the non-human antibody sequences in which the V_{H} and V_{L} are humanized by standard techniques (including CDR grafting) and optionally subsequent mutagenesis of certain amino acids in the framework region and/or the CDR. In one embodiment one to five amino acids (e.g. up to three) the framework region and/or one to three amino acids (e.g. up to two) in the CDRs can be modified by further mutations. For example the mutagenesis can be based upon molecular modeling as described by Riechmann, L., et al., Nature 332 (1988) 323-327 and Queen, C., et al., Proc. Natl. Acad. Sci. USA 86 (1989) 10029-10033, or others. The suited positions for such mutations can be identified e.g. by sequence or homology analysis, by choosing the human framework (fixed frameworks approach; homology matching or best-fit), by using consensus sequences, by selecting FRs from several different germlines, or by replacing non-human residues on the three dimensional surface with the most common residues found in human antibodies or based on sterical optimized interactions. In one embodiment such humanized variant is chimerized with a human constant region.

“Identity or homology” with respect to the sequence is defined herein as the percentage of amino acid residues in the candidate sequence that are identical with the parent sequence, after aligning the sequences and introducing gaps, if necessary, to achieve the maximum percent sequence identity. None of N-terminal, C-terminal, or internal extensions, deletions, or insertions into the antibody sequence shall be construed as affecting sequence identity or homology. The variant retains the ability to bind the variable domain of human TSLPR and preferably has properties, which are superior to those of the parent antibody. For example, the variant may have reduced side effects during treatment.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman, S., et al., J. Chromatogr. B 848 (2007) 79-87.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-TSLPR antibody” refers to one or more nucleic acid molecules encoding antibody heavy and/or light chains (or fragments thereof), including such nucleic acid molecule(s) in a single vector or separate vectors, and such nucleic acid molecule(s) present at one or more locations in a host cell.

The terms “nucleic acid” or “nucleic acid molecule”, as used herein, are intended to include DNA molecules and RNA molecules. A nucleic acid molecule may be single-stranded or double-stranded, but preferably is double-stranded DNA.

A nucleic acid is “operably linked” when it is placed into a functional relationship with another nucleic acid. For example, DNA for a presequence or secretory leader is operable linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are colinear, and, in the case of a secretory leader, contiguous and in reading frame. However, enhancers do not have to be contiguous. Linking is accomplished by ligation at convenient restriction sites. If such sites do not exist, synthetic oligonucleotide adaptors or linkers are used in accordance with conventional practice.

As used herein, the expressions “cell”, “cell line”, and “cell culture” are used interchangeably and all such designations include progeny. Thus, the words “transformants” and “transformed cells” include the primary subject cell and cultures derived there from without regard for the number of transfers. It is also understood that all progeny may not be precisely identical in DNA content, due to deliberate or inadvertent mutations. Variant progeny that have the same function or biological activity as screened for in the originally transformed cell are included.

The term “package insert” is used to refer to instructions customarily included in commercial packages of therapeutic products, that contain information about the indications, usage, dosage, administration, combination therapy, contraindications and/or warnings concerning the use of such therapeutic products.

An exemplary “parent” antibody comprises the CDR regions of antibody TSLP-012 and is preferably used for the preparation of the variant. Preferably, the parent antibody has a hamster framework region. The parent antibody may be subsequently humanized. Examples for humanized antibodies derived from TSLP-012 are TSLPR-012_{—}75, TSLPR-012_{—}141, TSLPR-012_{—}166, and TSLPR-012_{—}189.

The term “pharmaceutical formulation” (or pharmaceutical composition or pharmaceutical agent, which are used interchangeably herein) refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “specifically binding to TSLPR” as used herein means binding of the antibody to human TSLPR on TSLPR-transfected BA/F3 cells (ACC 300, DSMZ, http://www.dsmz.de) with an EC50 of at least 10⁻⁹ M⁻¹, preferably 10⁻⁹ M⁻¹ to 10⁻¹² M⁻¹ (FACS assay), but not to untransfected BA/F3 (no binding detectable at an antibody concentration of 10⁻⁷ M⁻¹).

Thymic stromal lymphopoietin receptor (TSLPR, TSLP receptor, CLRF2, SwissProt Q9HC73; SEQ ID NO:24) is a Type I cytokine receptor subunit which is closely related to the common cytokine γ chain (γc). TSLP is an interleukin (IL)-7-like cytokine that initiates and propagates allergic immune responses. TSLP is produced predominantly by epithelial cells and activated mast cells, and stimulates myeloid dendritic cells (mDC), which express a heterodimer consisting of the interleukin 7 (1-7) alpha chain and TSLP receptor. TSLP-activated mDC can promote naive CD4+ T cells to differentiate into a Th2 phenotype and can promote the expansion of CD4+ Th2 memory cells.

As used herein, “treatment” (and grammatical variations thereof such as “treat” or “treating”) refers to clinical intervention in an attempt to alter the natural course of the disease being treated, and can be performed either for prophylaxis or during the course of clinical pathology. Desirable effects of treatment include, but are not limited to, preventing occurrence or recurrence of the disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The “variable domain of an antibody” (variable domain of a light chain (V_{L}), variable domain of a heavy chain (V_{H})) as used herein denotes each pair of light and heavy chain domains which are involved directly in binding the antibody to the antigen. The variable light and heavy chain domains have the same general structure and each domain comprises four framework (FR) regions whose sequences are widely conserved, connected by three “hypervariable regions” (or complementary determining regions, CDRs). The framework regions adopt a β-sheet conformation and the CDRs may form loops connecting the 3-sheet structure. The CDRs in each chain are held in their three-dimensional structure by the framework regions and form together with the CDRs from the other chain the antigen binding site. The antibody's heavy and light chain CDR3 regions play a particularly important role in the binding specificity/affinity of the antibodies as described herein and therefore provide a further object of the invention.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operably linked. Such vectors are referred to herein as “expression vectors”.

The antibodies as described herein are preferably produced by recombinant means. Such methods are widely known in the state of the art and comprise protein expression in prokaryotic and eukaryotic cells with subsequent isolation of the antibody polypeptide and, usually, purification to a pharmaceutically acceptable purity. For the protein expression nucleic acids encoding light and heavy chains or fragments thereof are inserted into expression vectors by standard methods. Expression is performed in appropriate prokaryotic or eukaryotic host cells, such as CHO cells, NS0 cells, SP2/0 cells, HEK293 cells, COS cells, yeast, or E. coli cells, and the antibody is recovered from the cells (from the supernatant or after cells lysis). Recombinant production of antibodies is well-known in the art and described, for example, in the review articles of Makrides, S. C., Protein Expr. Purif. 17 (1999) 183-202; Geisse, S., et al., Protein Expr. Purif. 8 (1996) 271-282; Kaufman, R. J., Mol. Biotechnol. 16 (2000) 151-161; Werner, R. G., Drug Res. 48 (1998) 870-880. The antibodies may be present in whole cells, in a cell lysate, or in a partially purified or substantially pure form. Purification is performed in order to eliminate other cellular components or other contaminants, e.g., other cellular nucleic acids or proteins, by standard techniques, including, column chromatography and others well known in the art (see Ausubel, F., et al., ed. Current Protocols in Molecular Biology, Greene Publishing and Wiley Interscience, New York (1987)). Expression in NS0 cells is described by, e.g., Barnes, L. M., et al., Cytotechnology 32 (2000) 109-123; Barnes, L. M., et al., Biotech. Bioeng. 73 (2001) 261-270. Transient expression is described by, e.g., Durocher, Y., et al., Nucl. Acids. Res. 30 (2002) E9. Cloning of variable domains is described by Orlandi, R., et al., Proc. Natl. Acad. Sci. USA 86 (1989) 3833-3837; Carter, P., et al., Proc. Natl. Acad. Sci. USA 89 (1992) 4285-4289; Norderhaug, L., et al., J. Immunol. Methods 204 (1997) 77-87. A preferred transient expression system (HEK 293) is described by Schlaeger, E.-J. and Christensen, K., Cytotechnology 30 (1999) 71-83, and by Schlaeger, E.-J., J. Immunol. Methods 194 (1996) 191-199. Monoclonal antibodies are suitably separated from the culture medium by conventional immunoglobulin purification procedures such as, for example, protein A-Sepharose, hydroxylapatite chromatography, gel electrophoresis, dialysis, or affinity chromatography. DNA and RNA encoding the monoclonal antibodies is readily isolated and sequenced using conventional procedures. The hybridoma cells can serve as a source of such DNA and RNA. Once isolated, the DNA may be inserted into expression vectors, which are then transfected into host cells, such as HEK 293 cells, CHO cells, or myeloma cells that do not otherwise produce immunoglobulin protein, to obtain the synthesis of recombinant monoclonal antibodies in the host cells. Antibodies obtainable from said cell lines are preferred embodiments of the invention.

Amino acid sequence variants of a human anti-TSLPR antibody are prepared by introducing appropriate nucleotide changes into the antibody encoding DNA, or by peptide synthesis. Such modifications can be performed, however, only in a very limited range, e.g. as described above. For example, the modifications do not alter the above mentioned antibody characteristics such as the IgG isotype and epitope binding, but may improve the yield of the recombinant production, protein stability, or facilitate the purification. Any cysteine residue not involved in maintaining the proper conformation of the anti-TSLPR antibody may also be substituted, generally with serine, to improve the oxidative stability of the molecule and to prevent aberrant crosslinking. Conversely, cysteine bond(s) may be added to the antibody to improve its stability (particularly where the antibody is an antibody fragment such as an Fv fragment). Another type of amino acid variant of the antibody alters the original glycosylation pattern of the “parent” antibody. By “altering” is meant removing one or more carbohydrate moieties found in the antibody and/or adding one or more glycosylation sites that are not present in the “parent” antibody. Glycosylation of antibodies is typically N-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. Addition of glycosylation sites to the antibody is conveniently accomplished by altering the amino acid sequence such that it contains one or more of the above-described tripeptide sequences (for N-linked glycosylation sites).

Nucleic acid molecules encoding amino acid sequence variants of anti-TSLPR antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of humanized anti-TSLPR antibody.

Another type of covalent modification of the antibody comprises linking the antibody to one of a variety of non proteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192; 4,179,337.

The heavy and light chain variable domains as described herein are combined with sequences of promoter, translation initiation, constant region, 3′ untranslated region, polyadenylation, and transcription termination to form expression vector constructs. The heavy and light chain expression constructs can be combined into a single vector, co-transfected, serially transfected, or separately transfected into host cells which are then fused to form a single host cell expressing both chains.

In another aspect, there is provided a composition, e.g., a pharmaceutical composition, containing one or a combination of monoclonal antibodies, or the antigen-binding portion thereof, of the present invention, formulated together with a pharmaceutically acceptable carrier. As used herein, “pharmaceutically acceptable carrier” includes any and all solvents, dispersion media, coatings, antibacterial and antifungal agents, isotonic and absorption/resorption delaying agents, and the like that are physiologically compatible. Preferably, the carrier is suitable for injection or infusion. A pharmaceutical composition of the present invention can be administered by a variety of methods known in the art. As will be appreciated by the skilled artisan, the route and/or mode of administration will vary depending upon the desired results. Pharmaceutically acceptable carriers include sterile aqueous solutions or dispersions and sterile powders for the preparation of sterile injectable solutions or dispersion. The use of such media and agents for pharmaceutically active substances is known in the art. In addition to water, the carrier can be, for example, an isotonic buffered saline solution. Regardless of the route of administration selected, the antibodies as described in, which may be used in a suitable hydrated form, and/or the pharmaceutical compositions of the present invention, are formulated into pharmaceutically acceptable dosage forms by conventional methods known to those of skill in the art. Actual dosage levels of the active ingredients in the pharmaceutical compositions may be varied so as to obtain an amount of the active ingredient which is effective to achieve the desired therapeutic response for a particular subject, composition, and mode of administration, without being toxic to the subject (effective amount). The selected dosage level will depend upon a variety of pharmacokinetic factors including the activity of the particular compositions of the present invention employed, the route of administration, the time of administration, the rate of excretion of the particular compound being employed, other drugs, compounds and/or materials used in combination with the particular compositions employed, the age, sex, weight, condition, general health and prior medical history of the subject being treated, and like factors well known in the medical arts.

The invention comprises the use of the antibodies as described herein for the treatment of a subject suffering from an immunological.

The invention further provides a method for the manufacture of a pharmaceutical composition comprising an effective amount of an antibody as described herein together with a pharmaceutically acceptable carrier and the use of the antibody as described herein for such a method. The invention further provides the use of an antibody as described herein in an effective amount for the manufacture of a pharmaceutical agent, preferably together with a pharmaceutically acceptable carrier, for the treatment of a subject suffering from an immunological disease, especially from asthma, atopic dermatitis or rheumatoid arthritis.

also In an aspect, there is provided the use of an antibody as described herein in an effective amount for the manufacture of a pharmaceutical agent, preferably together with a pharmaceutically acceptable carrier, for the treatment of a subject suffering from an immunological disease.

Recombinant Methods and Compositions

Antibodies may be produced using recombinant methods and compositions, e.g., as described in U.S. Pat. No. 4,816,567. In one embodiment, isolated nucleic acid encoding an anti-TSLPR antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-TSLPR antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-TSLPR antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237; 5,789,199 and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, T. U., Nat. Biotech. 22 (2004) 1409-1414, and Li, H., et al., Nat. Biotech. 24 (2006) 210-215.

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177; 6,040,498; 6,420,548; 7,125,978 and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham, F. L., et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather, J. P., et al., Annals N.Y. Acad. Sci. 383 (1982) 44-68; MRC 5 cells; and FS4 cells. Other useful mammalian host cell lines include Chinese hamster ovary (CHO) cells, including DHFR-CHO cells (Urlaub, G., et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. J., and Wu, A. M., Methods in Molecular Biology, Antibody Engineering: Methods and Protocols, Vol. 248 (B. K. C. Lo, (ed.), Humana Press, Totowa, N.J.), (2004) pp. 255-268.

Pharmaceutical Formulations

Pharmaceutical formulations, pharmaceutical compositions or pharmaceutical agents of an anti-TSLPR antibody as described herein are prepared by mixing such antibody having the desired degree of purity with one or more optional pharmaceutically acceptable carriers (Osol, A., (ed.) Remington's Pharmaceutical Sciences, 16th edition, (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO 2006/044908, the latter formulations including a histidine-acetate buffer.

The formulation herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Osol, A., (ed.) Remington's Pharmaceutical Sciences, 16th edition, (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

Therapeutic Methods and Compositions

Any of the anti-TSLPR antibodies provided herein may be used in therapeutic methods.

In one aspect, an anti-TSLPR antibody for use as a medicament is provided. In further aspects, an anti-TSLPR antibody for use in treating a disease, preferably an immunological disease (especially asthma, atopic dermatitis or rheumatoid arthritis) characterized in being mediated by TSLPR activation is provided. In certain embodiments, an anti-TSLPR antibody for use in a method of treatment is provided. In certain embodiments, the invention provides an anti-TSLPR antibody for use in a method of treating an individual having a disease(s), preferably an immunological disease (especially asthma, atopic dermatitis or rheumatoid arthritis) characterized in being mediated by TSLPR activation comprising administering to the individual an effective amount of the anti-TSLPR antibody. In one such embodiment, the method further comprises administering to the individual an effective amount of at least one additional therapeutic agent, e.g., as described below. In further embodiments, the invention provides an anti-TSLPR antibody for use in diseases mediated by TSLPR activation. In certain embodiments, the invention provides an anti-TSLPR antibody for use in a method of treating diseases mediated by TSLPR activation in an individual comprising administering to the individual an effective of the anti-TSLPR antibody to mediate TSLPR activation, e.g., blocking TSLPR activation. An “individual” according to any of the above embodiments is preferably a human.

In a further aspect, the invention provides for the use of an anti-TSLPR antibody in the manufacture or preparation of a medicament. In one embodiment, the medicament is for treatment of a disease, preferably an immunological disease (especially asthma, atopic dermatitis or rheumatoid arthritis). In a further embodiment, the medicament is for use in a method of treating a disease, preferably an immunological disease (especially asthma, atopic dermatitis or rheumatoid arthritis) comprising administering to an individual having such disease an effective amount of the medicament. In one such embodiment, the method further comprises administering to the individual an effective amount of at least one additional therapeutic agent, e.g., as described below. In a further embodiment, the medicament is for use in a method of in an individual comprising administering to the individual an amount effective of the medicament to mediate TSLPR activation. An “individual” according to any of the above embodiments may be a human.

In a further aspect, the invention provides a method for treating an immunological disease (especially asthma, atopic dermatitis or rheumatoid arthritis). In one embodiment, the method comprises administering to an individual having such an immunological disease (especially asthma, atopic dermatitis or rheumatoid arthritis) an effective amount of an anti-TSLPR antibody. In one such embodiment, the method further comprises administering to the individual an effective amount of at least one additional therapeutic agent, as described below. An “individual” according to any of the above embodiments may be a human.

In a further aspect, the invention provides a method for administering to the individual an effective amount of an anti-TSLPR antibody as provided herein to treat an immunological disease (especially asthma, atopic dermatitis or rheumatoid arthritis). In one embodiment, an “individual” is a human.

In a further aspect, the invention provides pharmaceutical formulations comprising any of the anti-TSLPR antibodies provided herein, e.g., for use in any of the above therapeutic methods. In one embodiment, a pharmaceutical formulation comprises any of the anti-TSLPR antibodies provided herein and a pharmaceutically acceptable carrier. In another embodiment, a pharmaceutical formulation comprises any of the anti-TSLPR antibodies provided herein and at least one additional therapeutic agent, e.g., as described below.

Antibodies of the invention can be used either alone or in combination with other agents in a therapy. For instance, an antibody of the invention may be co-administered with at least one additional therapeutic agent.

Such combination therapies noted above encompass combined administration (where two or more therapeutic agents are included in the same or separate formulations), and separate administration, in which case, administration of the antibody of the invention can occur prior to, simultaneously, and/or following, administration of the additional therapeutic agent and/or adjuvant.

An antibody of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the invention would be formulated, dosed, and administered in a fashion consistent with good medical practice. Factors for consideration in this context include the particular disorder being treated, the particular mammal being treated, the clinical condition of the individual subject, the cause of the disorder, the site of delivery of the agent, the method of administration, the scheduling of administration, and other factors known to relevant medical practitioners. The antibody need not be, but may be optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the subject's clinical history and response to the antibody, and the discretion of the attending physician. The antibody is suitably administered to the subject at one time or over a series of treatments. Depending on the type and severity of the disease, about 1 μg/kg to 15 mg/kg (e.g. 0.1 mg/kg-10 mg/kg) of antibody can be an initial candidate dosage for administration to the subject (based on the subject's weight), whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the subject. Such doses may be administered intermittently, e.g. every week or every three weeks (e.g. such that the subject receives from about two to about twenty, or e.g. about six doses of the antibody). An initial higher loading dose, followed by one or more lower doses may be administered. The progress of this therapy is easily monitored by conventional techniques and assays.

It is understood that any of the above formulations or therapeutic methods may be carried out using an immunoconjugate of, in place of or in addition to an anti-TSLPR antibody as provided herein.

Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate of the invention in place of or in addition to an anti-TSLPR antibody.

The following are examples of methods and compositions of the invention. It is understood that various other embodiments may be practiced, given the general description provided above.

Description of the Sequence Listing

SEQ ID NO:1 sequence TSLPR-012 (hamster)HC variable region

SEQ ID NO:2 hCDR1 TSLPR-012

SEQ ID NO:3 hCDR2 TSLPR-012

SEQ ID NO:4 hCDR3 TSLPR-012

SEQ ID NO:5 sequence TSLPR-012 (hamster)LC variable region

SEQ ID NO:6 lCDR1 TSLPR-012

SEQ ID NO:7 lCDR2 TSLPR-012

SEQ ID NO:8 lCDR3 TSLPR-012

SEQ ID NO:9 sequence TSLPR-012_{—}141 (humanized) HC variable region

SEQ ID NO:2 hCDR1 TSLPR-012_{—}141

SEQ ID NO:10 hCDR2 TSLPR-012_{—}141

SEQ ID NO:4 hCDR3 TSLPR-012_{—}141

SEQ ID NO:11 sequence TSLPR-012_{—}141 (humanized) LC variable region

SEQ ID NO:12 lCDR1 TSLPR-012_{—}141

SEQ ID NO:13 lCDR2 TSLPR-012_{—}141

SEQ ID NO:8 lCDR3 TSLPR-012_{—}141

SEQ ID NO:9 sequence TSLPR-012_{—}75 (humanized) HC variable region

SEQ ID NO:2 hCDR1 TSLPR-012_{—}75

SEQ ID NO:10 hCDR2 TSLPR-012_{—}75

SEQ ID NO:4 hCDR3 TSLPR-012_{—}75

SEQ ID NO:14 sequence TSLPR-012_{—}75 (humanized) LC variable region

SEQ ID NO:12 lCDR1 TSLPR-012_{—}75

SEQ ID NO:15 lCDR2 TSLPR-012_{—}75

SEQ ID NO:8 lCDR3 TSLPR-012_{—}75

SEQ ID NO:16 sequence TSLPR-012_{—}166 (humanized) HC variable region

SEQ ID NO:17 hCDR1 TSLPR-012_{—}166

SEQ ID NO:10 hCDR2 TSLPR-012_{—}166

SEQ ID NO:4 hCDR3 TSLPR-012_{—}166

SEQ ID NO:18 sequence TSLPR-012_{—}166 (humanized) LC variable region

SEQ ID NO:12 lCDR1 TSLPR-012_{—}166

SEQ ID NO:15 lCDR2 TSLPR-012_{—}166

SEQ ID NO:8 lCDR3 TSLPR-012_{—}166

SEQ ID NO:9 sequence TSLPR-012_{—}189 (humanized) HC variable region

SEQ ID NO:2 hCDR1 TSLPR-012_{—}189

SEQ ID NO:10 hCDR2 TSLPR-012_{—}189

SEQ ID NO:4 hCDR3 TSLPR-012_{—}189

SEQ ID NO:19 sequence TSLPR-012_{—}189 (humanized) LC variable region

SEQ ID NO:12 lCDR1 TSLPR-012_{—}189

SEQ ID NO:7 lCDR2 TSLPR-012_{—}189

SEQ ID NO:8 lCDR3 TSLPR-012_{—}189

SEQ ID NO:20 sequence human Ig gamma1 (G1M1,17)

SEQ ID NO:22 sequence human Ig gamma4

SEQ ID NO:22 sequence human Ig gamma4 (S288P,L235E)

SEQ ID NO:23 sequence human Ig kappa

SEQ ID NO:24 human TSLPR

SEQ ID NO:25 cynomolgous TSLPR

### EXAMPLE 1

Description of Immunization

Immunization of Armenian Hamsters with Human TSLPR

Armenian hamsters were immunized with 50 μg recombinant human soluble TSLPR-Fc fusion protein (TSLPR amino acids 23 to 231 of SEQ ID NO:24) at day 0 with complete Freund's adjuvant and at days 28, 56, 84 and 112 with incomplete Freund's adjuvant by intraperitoneal injection. Blood was taken at days 91 and 119 and serum prepared, which was used for titer determination by ELISA and proliferation assay (see below). Animals with highest titers were selected for boosting at day 140 by intravenous injection of 50 μg of recombinant human soluble TSLPR-Fc fusion protein and antibodies were isolated. Antibodies as described herein are identified by binding to human TSLPR, binding to BA/F3-TSLPR/IL7Ralpha and Cyno-TSLPR-HEK293 cells in a FACS assay.

### EXAMPLE 2

Binding to Human TSLPR (ELISA)

Binding of anti-TSLPR antibodies to human TSLPR was determined by ELISA. Human recombinant TSLPR-Fc were immobilized on a 384-well Nunc Maxisorp™ plate at 150 ng/ml, 25 μl/well, in PBS, by incubation overnight at 2-8° C. After four washes with 90 μl PBST (0.1% Tween® 20 in PBS) per well, blocking of the plate with PBS/1% BSA (blocking buffer) for 1 h at room temperature was followed by four wash steps (PBST) and incubation with anti-TSLPR antibodies at different concentrations in blocking buffer or hybridoma supernatants for 1 h at room temperature. After further four washes, antibodies were detected with anti-hamster-HRP antibody (Dianova, Hamburg, Germany, #127-035-160) diluted 1:5000 in blocking buffer, for 1 h at room temperature. Signal was developed by addition of ABTS® (Roche Diagnostics GmbH) for 10-30 minutes after another four wash steps. Absorbance was read out at 405 nm.

### EXAMPLE 3

Binding to BA/F3-TSLPR/IL7Rα and Cyno-TSLPR-HEK293 cells (FACS)

Binding of anti-TSLPR antibodies to TSLPR-expressing cells was demonstrated by FACS using BA/F3 cells stably overexpressing human TSLPR and IL-7Rα; binding to TSLPR from Cynomolgous monkey was demonstrated using HEK293 cells transiently transfected with Cynomolgous TSLPR (SEQ ID NO:25). BA/F3-TSLPR/IL7Rα or Cyno-TSLPR-HEK293 cells were starved from TSLP overnight, harvested by centrifugation, washed once in PBS and re-suspended in FACS buffer (PBS/5% FCS) at 10⁶/ml. 100 μl of cell suspension were incubated in a 96-well round-bottom plate well together with 100 μl of anti-TSLPR antibody solution for 45 minutes on ice. After three washes with FACS buffer, cells were re-suspended in a solution of PE-coupled anti-hamster-F(ab)2 (Dianova, Hamburg, Germany, #127-115-160) (1:200 in FACS buffer) and incubated for 30 minutes on ice. After three further washes cells were re-suspended in 200 μl of FACS buffer and measured on a FACSCanto™ flow cytometer (10,000 cells, flow rate 3).

### EXAMPLE 4

Blocking of BA/F3-TSLPR/IL7Rα Proliferation

30 μl of hybridoma supernatant or anti-TSLPR antibody solution (antibodies as described herein and antibody 1D6.C9 from WO 2007/112146) were added to a well of a white 384-well plate, followed by addition 20 μl of BA/F3 cells stably overexpressing human TSLPR and IL-7Rα, suspended at a density of 25,000 cells/ml in TSLP-free growth medium (RPMI/10% FCS). After 1 h of incubation at 37° C./5% CO₂, 10 μl of TSLP solution were added to a final concentration of 1 ng/ml. The cells were incubated for 48 h at 37° C./5% CO₂, then 20 μl of CellTiter-Glo® reagent mix were added per well and luminescence read out after 2 min of shaking.

### EXAMPLE 5

Blocking of TSLP/TSLPR/IL7Rα Interaction in ELISA

Recombinant human TSLPR-Fc was coated to 96-well Nunc Maxisorp™ plate wells by incubating 50 μl of a 0.2 μg/ml solution of TSLPR-Fc in PBS per well for 1 h at RT. Subsequently the wells were blocked with 1% Crotein™ C in PBS (blocking buffer) for 1 h at RT, followed by two washes with wash buffer (ice-cold PBS/0.05% TWEEN® 20). 50 μl of anti-TSLPR antibody solution (antibodies and according to WO 2007/112146) were then added to the wells and incubated for 2 h at 4° C., followed by four wash steps. After addition of 50 μl of a mixture of human recombinant TSLP (12.5 ng/ml) and human recombinant IL7Rα-Fc (1 μg/ml) in blocking buffer, the plate was shaken for 1 h at 4° C., followed by further four wash steps. TSLP binding was detected by incubation with 50 μl of biotinylated anti-TSLP antibody (0.08 μg/ml in blocking buffer), followed by four washes and incubation with 50 μl of streptavidin-HRP (0.25 μg/ml in blocking buffer) per well. After another four washing steps, color was developed by incubation with 50 μl of 3,3,5,5-Tetramethylbenzidine substrate, followed by addition of 50 μl of 1 N HCl. Absorbance was read out at 450 and 620 nm.

### EXAMPLE 6

Affinity Determination of the Antibody-TSLPR Complexes Using Biacore

The binding properties of monoclonal anti-TSLPR antibodies and according to WO 2007/112146 were analyzed by surface plasmon resonance (SPR) technology using a Biacore 3000 instrument. The affinity was determined using an assay setup with capturing antibodies. These capturing antibodies (goat anti-human-IgG, goat anti-hamster-IgG) were immobilized on the surface of a CM5 biosensorchip using amine coupling chemistry. The capturing antibodies were injected in sodium acetate buffer, pH 5.0 at a concentration of 5 μg/ml aiming for a surface density of approx 1000 RU. Remaining reactive groups were inactivated by an injection of 1 M ethanolamine/HCl pH 8.5. Running buffer was HBS-P. The anti-TSLPR antibodies (analyte 1) were diluted in HBS-P to a concentration of 25 nM and injected at a flow rate of 5 μl/min for 3 minutes, resulting in a binding signal of 100-400RU dependent on the antibodies applied. The extracellular domain of TSLPR, TSLPR-ECD, obtained by cleavage of a TSLPR-Fc fusion protein expressed in HEK cells (analyte 2), was injected in concentrations ranging from 0-50 nM at a flow rate of 20 μl/min. The contact time (association phase) was 3 min for TSLPR followed by 5 minutes of dissociation. Regeneration solution 10 mM glycine HCl pH1.5 or 10 mM glycine HCl pH1.7 was injected twice at a flow rate of 10 μl/min for 1 min to remove any bound protein after each binding cycle. All interactions were performed at 25° C. (standard temperature). Signals were detected at a detection rate of 1 signal/second. The signals from a reference flow cell (FC1: same capturing antibody and addition of control antibody) and from blank buffer injections were subtracted and data were evaluated. All binding curves were fitted to a 1:1 Langmuir binding model. Association rate constant (ka), dissociation rate constant (kd) and the dissociation equilibrium constant (K_{D}) were calculated accordingly.

### EXAMPLE 7

Inhibition of CD11c+ DC TARC Secretion

Secretion of TARC/CCL17 and related cytokines by CD11c+ dendritic cells (DCs) is an indicator of DC activation by TSLP, finally leading to TH2 differentiation in asthma and atopic dermatitis. Blocking of TARC secretion by anti-TSLPR antibodies and including 1D6.C9 (WO 2007/112146) was measured using an ELISA on supernatants from TSLP-activated CD11c+ DCs. CD11c+ DCs were prepared essentially as described in Soumelis et al., Nature Immunol. 7, 673-680 (2002) from peripheral blood mononuclear cells (PBMCs) by depletion of cells expressing the lineage markers CD3, CD14, CD19, CD56 and glycophorin A using magnetic beads, followed by FACS-sorting of CD11c+ cells. DCs were cultured in RPMI with 10% FCS, 1% pyruvate, 1% HEPES and penicillin/streptomycin, pre-incubated with anti-TSLPR antibodies at different concentrations and stimulated with 15 ng/ml TSLP for 48 hours. TARC was measured in DC supernatants using a commercially available human TARC ELISA. All antibody concentrations mentioned in this patent application relate to an antibody molecular weight of 150.000.

Results of examples 4, 5, 6 and 7 are shown in Table 1. Results from example 3 are shown in table 2.

Inhibition of TSLP-induced CD80, CD86, MDC, Eotaxin, Eotaxin 3, MIP-1β and IP10 was also observed (Data not shown)

TABLE 1

| | IC50 | | BIAcore | IC50 DC |
|---|---|---|---|---|
| | proliferation | IC50 ELISA | affinity | assay |
| | (example 4) | (example5) | (example 6) | (example 7) |
| Antibody | [ng/ml] | [ng/ml] | K_{D} [10⁻⁹ M] | [ng/ml] |
| 1D6.C9 | 84 | 15.2 | 2.97 | n.d. |
| (WO2007/ | | | | |
| 112146) | | | | |
| TSLPR- | 26 | 13.4 | 0.50 | 42 |
| 012_75, | | | | |
| CDR | | | | |
| grafted | | | | |
| TSLPR- | 37 | 10.4 | 0.87 | 46 |
| 012_141, | | | | |
| CDR | | | | |
| grafted | | | | |
| TSLPR- | 34 | 12.2 | 2.45 | 86 |
| 012_166, | | | | |
| CDR | | | | |
| grafted | | | | |
| TSLPR- | 25 | 13.6 | 0.60 | 23 |
| 012_189, | | | | |
| CDR | | | | |
| grafted | | | | |
| TSLPR-012 | 26 | 8.8 | 0.25 | 129 |

TABLE 2
(example 3)

| | EC50 FACS | EC50 FACS |
|---|---|---|
| | BA/F3- | HEK-Cyno- |
| | TSLPR/IL7Rα | TSLPR |
| Antibody | [ng/ml] | [ng/ml] |
| TSLPR- | 15.7 | 10.8 |
| 012_75, | | |
| CDR grafted | | |
| TSLPR- | 13.3 | 9.7 |
| 012_141, | | |
| CDR grafted | | |
| TSLPR- | 11.8 | 8.6 |
| 012_166, | | |
| CDR grafted | | |
| TSLPR- | 14.9 | 10.3 |
| 012_189, | | |
| CDR grafted | | |
| TSLPR-012 | 15.6 | 11.8 |

### EXAMPLE 8

Inhibition of Dendritic Cell (DC) Activation

The ability of the antibodies described herein to block dendritic cell activation, including TARC, MDC, IL-8 secretion and CD80 and CD86 on the cell surface was tested. For this purpose human dendritic cells were isolated from peripheral blood (whole blood) and stimulated with 10 ng/ml of TSLP in the presence or absence of the antibody. After 48 hours of incubation, cytokines and surface markers were measured. The antibody produced a concentration-dependent inhibition of the responses. For TARC, for example, for anti-TSLPR antibody TSLPR-012_{—}141 the average IC50±SE was 2.4 nM. For anti-TSLPR antibody TSLPR-012_{—}141, the range of IC50s for all responses was between 1.7 and 5.9 nM

### EXAMPLE 9

Inhibition of Th2 Cytokine Production in Polarization Assay

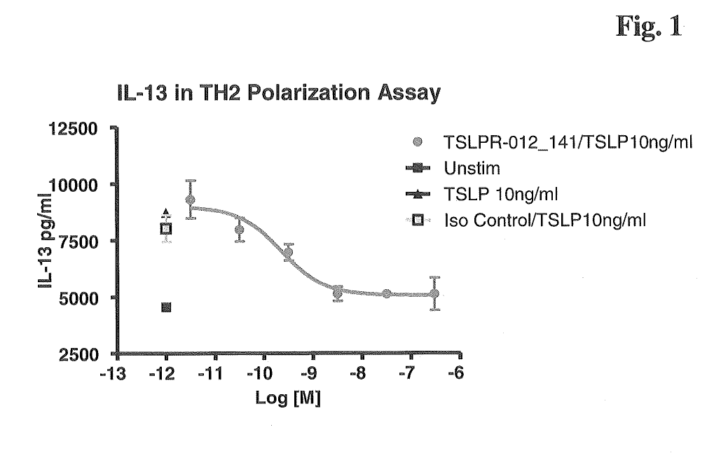

DCs activated with TSLP have been shown to promote differentiation of naive T cells into the Th2 phenotype. To test the ability of the antibodies provided herein to block this process, DCs were stimulated with TSLP in the presence and absence of antibody. Then they were co-cultured with naive T-cells under Th2 differentiation conditions (i.e. under conditions where secretion of Th2 cytokines can be observed). When naive T cells were cultured with TSLP-activated DCs, secretion of Th2 cytokines, IL-13 and IL-5, were observed. When the activation of the DCs was done in the presence of different concentrations of the antibody, a dose dependent inhibition of IL-13 (see FIG. 1) and IL-5 production was observed. The IC50 of anti-TSLPR antibody TSLPR-012_{—}141 for IL-13 release was 2.57 nM. For IL-5 release the IC50 was 1.62 nM.

### EXAMPLE 10

Inhibition of Mast Cell Cytokine Release

The ability of the anti-TSLPR antibodies described herein to inhibit TSLP-induced mast cell cytokine production was tested using human mast cell cultures derived from CD34+ hematopoietic stem cells. These cells were cultured for 8 weeks and stimulated with IL-4 for 5 days before the assay was performed. Cells were incubated with different concentrations of antibody or antibody control for 1 hour and then stimulated with TSLP in combination with IL-1β/TNFα or IL-33. IL-13, IL-5 and GMCSF were the read-outs for the assay. Anti-TSLPR antibody TSLPR-012_{—}141 potently inhibited cytokine production by mast cells when stimulated with TSLP in combination with either IL-1β/TNFα or IL-33. The experiment was repeated with 3 different cultures from different donors. The average IC50s for these experiments ranged from 0.04 to 0.06 nM.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. An antibody specifically binding to human thymic stromal lymphopoietin receptor (TSLPR), comprising a) a heavy chain variable domain comprising a CDR1 region of SEQ ID NO: 2, a CDR2 region of SEQ ID NO:3, and CDR3 region of SEQ ID NO:4, and b) a light chain variable domain comprising a CDR1 region of SEQ ID NO: 6, a CDR2 region of SEQ ID NO:7, and a CDR3 region of SEQ ID NO:8.

2. The antibody according to claim 1 comprising a heavy chain variable domain comprising SEQ ID NO:1 and a light chain variable domain comprising SEQ ID NO:5.

3. The antibody according to claim 1, wherein said antibody is a chimeric or humanized variant thereof.

4. The antibody according to claim 3, wherein said antibody comprising a) a heavy chain variable domain comprising a CDR1 region of SEQ ID NO: 2 or 17, a CDR2 region of SEQ ID NO:3 or 10, and CDR3 region of SEQ ID NO:4, and b) a light chain variable domain comprising a CDR1 region of SEQ ID NO: 6 or 12, a CDR2 region of SEQ ID NO:7, 13 or 15, and a CDR3 region of SEQ ID NO:8.

5. The antibody according to claim 3, wherein said antibody has
a) a heavy chain variable domain comprising a CDR1 region of SEQ ID NO: 2, a CDR2 region of SEQ ID NO:10, and CDR3 region of SEQ ID NO:4, and a light chain variable domain comprising a CDR1 region of SEQ ID NO: 12, a CDR2 region of SEQ ID NO:13 and a CDR3 region of SEQ ID NO:8;
b) a heavy chain variable domain comprising a CDR1 region of SEQ ID NO: 2, a CDR2 region of SEQ ID NO:10, and CDR3 region of SEQ ID NO:4, and a light chain variable domain comprising a CDR1 region of SEQ ID NO: 12, a CDR2 region of SEQ ID NO:15 and a CDR3 region of SEQ ID NO:8;
c) a heavy chain variable domain comprising a CDR1 region of SEQ ID NO: 17, a CDR2 region of SEQ ID NO:10, and CDR3 region of SEQ ID NO:4, and a light chain variable domain comprising a CDR1 region of SEQ ID NO: 12, a CDR2 region of SEQ ID NO:15 and a CDR3 region of SEQ ID NO:8; or
d) a heavy chain variable domain comprising a CDR1 region of SEQ ID NO: 2, a CDR2 region of SEQ ID NO:10, and CDR3 region of SEQ ID NO:4, and a light chain variable domain comprising a CDR1 region of SEQ ID NO: 12, a CDR2 region of SEQ ID NO:7 and a CDR3 region of SEQ ID NO:8.

6. An antibody according to claim 3, wherein said antibody has
a) a heavy chain variable domain comprises SEQ ID NO:9 and a light chain variable domain comprises SEQ ID NO:11;
b) a heavy chain variable domain comprises SEQ ID NO:9 and a light chain variable domain comprises SEQ ID NO:14;
c) a heavy chain variable domain comprises SEQ ID NO:16 and a light chain variable domain comprises SEQ ID NO:18; or
d) a heavy chain variable domain comprises SEQ ID NO:9 and a light chain variable domain comprises SEQ ID NO:19.

7. An antibody according to claim 1, wherein said antibody is a human IgG1 or IgG4 isotype.

8. An antibody according to claim 1, wherein said antibody is a human IgG4 isotype with a substitution of serine 228 to proline and lysine 235 to glutamic acid, wherein the amino acid residue positions are numbered according to Kabat.

9. An antibody according to claim 1, wherein said antibody is a human IgG1 isotype with a substitution of lysine 234 to alanine and lysine 235 to alanine, wherein the amino acid residue positions are numbered according to Kabat.

10. A nucleic acid encoding an antibody according to claim 1 that specifically binds to human thymic stromal lymphopoietin receptor (TSLPR).

11. An expression vector comprising a nucleic acid according to claim 10.

12. A host cell comprising the nucleic acid of claim 10.

13. A method for the production of a recombinant human or humanized antibody according to claim 1, comprising the steps of expressing a nucleic acid according claim 10 in a prokaryotic or eukaryotic host cell and recovering said antibody from said cell or the cell culture supernatant.

14. A pharmaceutical composition, wherein said composition comprises an antibody according to claim 1.

15. Method for the manufacture of a pharmaceutical composition comprising an antibody according to claim 1.

16. Method for the manufacture of a medicament for the treatment of diseases, characterized in comprising an antibody specifically binding to human TSLPR according to claim 1.

17. Use of an antibody according to claim 1 for the manufacture of a pharmaceutical composition.

18. Use of an antibody according to any one of claim 1 for the manufacture of a medicament.

19. Use of an antibody according to claim 1 for the treatment of a disease.

20. Use according to claim 19, wherein the disease is an immunological disease.

21. An antibody according to claim 1 for use in the treatment of a disease.

22. An antibody according to claim 21, wherein the disease is an immunological disease.

23. A method of treatment of a patient suffering from a disease, said treatment comprising administering to the patient an antibody according to claim 1.

24. A method of treatment according to claim 23, wherein said disease is an immunological disease.

25. A host cell according to claim 12, wherein said host cell is prokaryotic or eukaryotic.

26. A host cell according to claim 12, wherein said host cell is prokaryotic.

27. A host cell according to claim 12, wherein said host cell is eukaryotic.


SEQUENCE LISTING (25 sequences)

SEQ ID NO: 1 - protein, 122 aa, Cricetulus migratorius
QIQLQESGPGLVKPSQSLSLTCSVTGFSITTSGYYWTWIRQFPGKKLEWMGYIGYNSKTYYNPSLKSRISITRDTSKNQFLLHLNSVTTEDTATYYCARSLYGGYKDAFDSWGQGTLVTVSS

SEQ ID NO: 2 - protein, 12 aa, Cricetulus migratorius
GFSITTSGYYWT

SEQ ID NO: 3 - protein, 16 aa, Cricetulus migratorius
YIGYNSKTYYNPSLKS

SEQ ID NO: 4 - protein, 12 aa, Cricetulus migratorius
SLYGGYKDAFDS

SEQ ID NO: 5 - protein, 108 aa, Cricetulus migratorius
DVVLTQTPATLSAIPGERVTMTCKASQSIGTSLHWYQHRPNETPRLLIKFASRSITGIPSRFSGSGSGTDFTLGINNLEAEDFAIYYCQQSPGFPPTFGSGTKLEINR

SEQ ID NO: 6 - protein, 11 aa, Cricetulus migratorius
KASQSIGTSLH

SEQ ID NO: 7 - protein, 7 aa, Cricetulus migratorius
FASRSIT

SEQ ID NO: 8 - protein, 9 aa, Cricetulus migratorius
QQSPGFPPT

SEQ ID NO: 9 - protein, 122 aa, Artificial
  sequence TSLPR-012_141 (humanized) HC variable region
QVQLVESGGGLVKPGGSLRLSCAASGFSITTSGYYWTWIRQFPGKGLEWMGYIGYNSKTYYNPALKSRITISRDNAKNSLYLQMNSLRAEDTAVYYCARSLYGGYKDAFDSWGQGTLVTVSS

SEQ ID NO: 10 - protein, 16 aa, Artificial
  hCDR2 TSLPR-012_141
YIGYNSKTYYNPALKS

SEQ ID NO: 11 - protein, 108 aa, Artificial
  sequence TSLPR-012_141 (humanized) LC variable region
DIQMTQSPSSLSASVGDRVTITCRASQSIGTSLHWYQQKPGKAPKLLIKFASRLQSGVPSRFSGSGSGTDFTLTISSLQPEDFATYYCQQSPGFPPTFGQGTKLEIKR

SEQ ID NO: 12 - protein, 11 aa, Artificial
  lCDR1 TSLPR-012_141
RASQSIGTSLH

SEQ ID NO: 13 - protein, 7 aa, Artificial
  lCDR2 TSLPR-012_141
FASRLQS

SEQ ID NO: 14 - protein, 108 aa, Artificial
  sequence TSLPR-012_75 (humanized) LC variable region
EIVLTQSPDFQSVTPKEKVTITCRASQSIGTSLHWYQQKPDQSPKLLIKFASRSISGVPSRFSGSGSGTDFTLTINSLEAEDAATYYCQQSPGFPPTFGQGTKLEIKR

SEQ ID NO: 15 - protein, 7 aa, Artificial
  lCDR2 TSLPR-012_75
FASRSIS

SEQ ID NO: 16 - protein, 122 aa, Artificial
  sequence TSLPR-012_166 (humanized) HC variable region
QVQLQESGPGLVKPSQTLSLTCTVSGFSITTSGYYWSWIRQHPGKGLEWIGYIGYNSKTYYSPSLKSRVTISRDTSKNQFSLKLSSVTAADTAVYYCARSLYGGYKDAFDSWGQGTLVTVSS

SEQ ID NO: 17 - protein, 12 aa, Artificial
  hCDR1 TSLPR-012_166
GFSITTSGYYWS

SEQ ID NO: 18 - protein, 108 aa, Artificial
  sequence TSLPR-012_166 (humanized) LC variable region
DIQMTQSPSSLSASVGDRVTITCRASQSIGTSLHWYQHRPGETPKLLIKFASRSISGVPSRFSGSGSGTDFTLTISSLQPEDFATYYCQQSPGFPPTFGQGTKLEIKR

SEQ ID NO: 19 - protein, 108 aa, Artificial
  sequence TSLPR-012_189 (humanized) LC variable region
EIVMTQSPATLSVSPGERATLSCRASQSIGTSLHWYQQKPGQAPRLLIKFASRSITGIPARFSGSGSGTEFTLTISSLQSEDFAVYYCQQSPGFPPTFGQGTKLEIKR

SEQ ID NO: 20 - protein, 330 aa, Homo sapiens [sequence omitted]

SEQ ID NO: 21 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 327 aa, Artificial
  sequence human Ig gamma4 (S228P,L235E)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 371 aa, Homo sapiens
MGRLVLLWGAAVFLLGGWMALGQGGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKFILISSLAILLMVSLLLLSLWKLWRVKKFLIPSVPDPKSIFPGLFEIHQGNFQEWITDTQNVAHLHKMAGAEQESGPEEPLVVQLAKTEAESPRMLDPQTEEKEASGGSLQLPHQPLQGGDVVTIGGFTFVMNDRSYVAL

SEQ ID NO: 25 - protein, 372 aa, Macaca fascicularis
MGRLVLLWGAAVFLLGGWMALGQVATGEGLQIQIMYFNLETVQVTWNASHYPRSNLSFHYKFSRDEAYDQCTVYILQEGHTSGCLLDAEQQDDILYFSIRNGTHPVFTASRWIFYYLKPSSPKQVSFSWHQDAVTLTCSDLSYRGLLYEVQYRSPFDTEWQSKQENTCNVTVEDLDAEKCYAFRARVKAMEDAYGPDTYPSDWSEVTCWQRGETRDSCPEPRTPPKPKLSKFMLVSSLAILLMVCLLLLSLRKLWRVKKFLMPSVPDPKSTFPGLFEIHQGNFQEWITDTQNVAHLHKIAGAEPEGGPEESLVVQLAKTEAESPRTPGPQTEDGKASGGSLLLPHQPPLQGGDVVTLGGFTFVMNDSSYVAL